(19) United States
(12) Patent Application Publication
(10) Pub. No.: US 2008/0226860 A1
(43) Pub. Date: Sep. 18, 2008

(54) CYANINE DYES AND USE THEREOF

(76) Inventor: Yen-Cheng TSAI, Taipei County (TW)

Correspondence Address: RABIN & Berdo, PC, 1101 14TH STREET, NW, SUITE 500, WASHINGTON, DC, 20005, US

(21) Appl. No.: 12/048,938

(22) Filed: Mar. 14, 2008

(30) Foreign Application Priority Data
Mar. 16, 2007 (TW) 96109075

(51) Int. Cl. B32B 3/00; C07D 209/56

(52) U.S. Cl. 428/64.8; 548/427

(57) ABSTRACT

The present invention relates to a recording medium consisting of transparent substrate, recording layer, reflective layer, and protective layer, wherein the recording layer comprising at least one organic optical dye of structural formula (1):

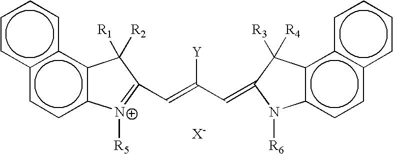

wherein each of R₁, R₂, R₃, R₄, R₅, R₆, X, and Y is defined as herein. The recording medium of present invention is useful as a write once recording medium, particularly is using as a recording medium for laser wavelength of 630 to 660 nm.

### FIELD OF THE INVENTION

The present invention relates to a recording medium, which uses a special cyanine dye in the recording layer. Particularly, the present invention relates to a write once recording medium suitable for laser wavelength of 630 to 660 nm.

### BACKGROUND OF THE INVENTION

In the past decade, there are great progresses in the development of recording medium, especially write once recording medium. The recording speed of Recordable Compact Disc, called as CD-R, is improved from 1×, 2×, 4× to 52×, even to 56×. However, the wavelength of laser light used in CD-R format is of 780 nm, thus a larger light spot is formed, and only about 700 MB capacity could be provided on a recording disc of 12 cm in diameter. In comparison with the recording media of next generation, which underline multifunctional requirement in video/audio entertainment, the capacity of such CD-R is obviously insufficient. Accordingly, a write once recording medium called as DVDR (DVD-R or DVD+R) using for shorter wavelength (of 630˜660 nm) to increase storage density was developed.

Organic dyes useful in the wavelength range of 630-660 nm have been published in many references. Among others, the azo-metal complex dyes are widely investigated and discussed, such as in U.S. Pat. Nos. 6,225,023, 6,284,877, 6,551,682, 6,794,114, and 6,815,033. Owing to the special chemical structure, this kind of metal complex has good light resistance. Azo-metal complex dyes have been commonly used in the recording layer of DVD for years, and can provide satisfying quality of recording under the burning speed of 1×-8×. However, azo-metal complex may confer higher burning power to the recording disc when the burning speed is required to 16× (or higher), because it has relatively higher heat-degradation temperature (280-380° C., as measured by thermogravimetric analysis (TGA)). The higher burning power may result in worse compatibility with different disc writer, and failed recording in certain high-speed recorders. Moreover, the recording layer of DVDR disc using such azo-metal complex usually causes lowered reflectivity, and higher possibility of failure in recording or reading on older CD-ROM drives.

Oxonol dyes have also been widely discussed, for example in U.S. Pat. Nos. 6,646,132, 6,225,024, 6,670,475, 6,020,105, and Taiwan Patent Nos 091132186 and 090118281. In comparison to commonly used azo-metal complex dyes, Oxonol dyes are promised to provide lower burning power and better light resistance. However, the insolubility of oxonol dye forces it to be dissolved in certain solvent with heating at manipulation, and easy to be precipitated out from a formulated solution, and thus change the concentration of dye solution, which is a great difficulty and challenge for disc production. Besides, oxonol dyes usually ask for the substrate with smaller groove depth (of about 120 nm), which is another problem in preparing stamper. Therefore, compared with the substrate with groove depth of 150˜180 nm used for other types of recording dye, the usage of oxonol dye in disc manufacture can not avoid the disadvantage of stringent processing condition and low production quality.

Cyanine dyes are another important choice for disc manufacture. Among others, the asymmetric cyanine dye has been widely discussed, for example in U.S. Pat. No. 6,413,607, and Taiwan Patent No 1241581. The common chemical structure of cyanine dye, represented by following formula (II) and (III), is consisted of two asymmetric substituted benzene or naphthalene rings, which are connected via central conjugated double bonds.

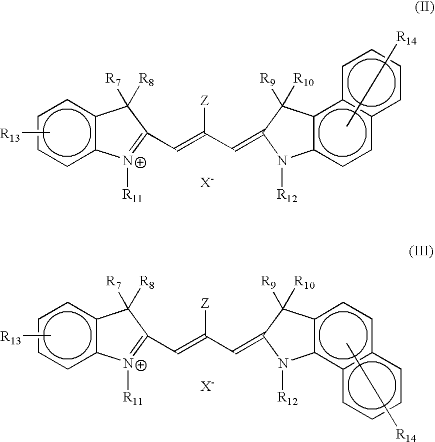

wherein R₇ to R₁₂ are commonly alkyl, R₁₃ and R₁₄ may be electron withdrawing group or electron donating group, Z often is a hydrogen atom or halogen atom, and X⁻ is a counter ion.

Another type of cyanine dye is focused on the counter ion (that is X⁻ in above formula (II) and (III)), and it is azo-metal complex anion to be the commonly used counter ion, such as described in U.S. Pat. Nos. 6,525,181 and 6,413,607, and Taiwan Patent No 090102535. These patents had tried to combine the high reflectivity of cyanine dye and the excellent light resistance of azo-metal complex. However, using the combined dyes in disc production substantially causes high parity of inner-code (PI), and it is a problem that still can't be dissolved by nowaday process. It is because that cyanine dye and azo-metal complex are suitable different kind of substrate with distinct groove geometry respectively. When these two dyes are combined by ionic binding, there appears serious contrary in groove geometry, which leads to a bad property of disc recoding.

Thus, the present provides a cyanine dye with special chemical structure, which has not only good solubility (as comparing to azo-metal complex) but also better (lower) burning power and higher reflectivity. The DVDR disc prepared with present dye can meet the requirement in high-speed (16× or higher) burning, and provides excellent compatibility with disc writers.

### SUMMARY OF THE INVENTION

In one aspect, the present invention provides an optical recording medium suitable for high-speed (16× or higher) DVD-R/+R burning, wherein the recording layer of the recording medium comprises at least one special cyanine dye.

In another aspect, the present invention provides a special cyanine dye for using in the recording layer of recording medium to meet the requirement in high-speed (16× or higher) DVD-R/+R burning.

The cyanine dye of present invention has following structural formula:

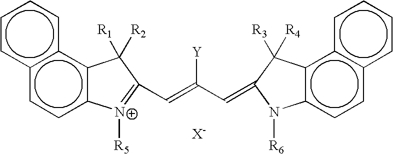

Wherein R₁, R₂, R₃, and R₄ may be different or identical, and each is unsubstituted or substituted C₁-C₆ alkyl, phenyl, benzyl, or alkylphenyl; R₅, and R₆ is unsubstituted or substituted C₁-C₄ alkyl, wherein the possible substituent is hydroxyl, alkylamino, alkylacyloxy (—OC(═O)R), alkylaminoacyloxy (—OC(═O)NHR), tosyloxy (—OSO₂C₆H₄CH₃), or tirfluoromethylsulfonyloxy (—OSO₂CF₃); Y is hydrogen, halogen, methyl, ethyl, phenyl, or alkylamino; and counter ion X⁻ is an anion selected from Cl⁻, Br⁻, I⁻, ClO₄⁻, BF₄⁻, PF₆⁻, or SbF₆⁻.

### DETAILED DESCRIPTION OF THE INVENTION

The present invention relates to an optical recording medium, which comprises a substrate, and an organic recording layer coated on the substrate, wherein the recording layer records information on it by the laser beam radiation of special wavelength (of 630˜660 nm), and comprises at least one cyanine dye of structural formula (I):

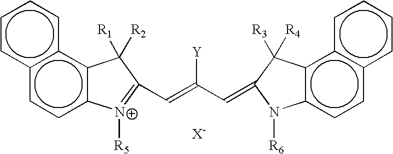

Wherein R₁, R₂, R₃, and R₄ may be different or identical, and each is unsubstituted or substituted C₁-C₆ alkyl, phenyl, benzyl, or alkylphenyl; R₅, and R₆ is unsubstituted or substituted C₁-C₄ alkyl, wherein the possible substituent is hydroxyl, alkylamino, alkylacyloxy (—OC(═O)R), alkylaminoacyloxy (—OC(═O)NHR), tosyloxy (—OSO₂C₆H₄CH₃), or tirfluoromethylsulfonyloxy (—OSO₂CF₃); Y is hydrogen, halogen, methyl, ethyl, phenyl, or alkylamino; and counter ion X⁻ is an anion selected from Cl⁻, Br⁻, I⁻, ClO₄⁻, BF₄⁻, PF₆⁻, or SbF₆⁻.

In the molecular design of common cyanine dyes, R₁, R₂, R₃, and R₄ usually are lower alkyl, most frequently R₁, R₂, R₃, and R₄ are all CH₃. However, such formed dyes could not meet the requirement in high-speed burning. It has reported that introducing an electron withdrawing group or an electron donating group on the benzene ring of asymmetric indoline may improve the insufficient sensitivity of cyanine dyes. Surprisingly, it is found by present invention that introducing at least two benzene rings into R₁, R₂, R₃, and R₄ can not only increase effective resonance number of conjugated double bond, but also increase the absorbance wavelength of dye molecule, and thus provide better sensitivity during recording, further decrease burning power of dyes to meet the requirement in high-speed burning and compatibility with disc writer. For accomplishing such purpose, at least two of R₁, R₂, R₃, and R₄ must be phenyl, benzyl, or tolyl.

R₅ and R₆ commonly are alkyl, and the carbon number of such alkyl may influence the final solubility of cyanine dye. In general, the more carbon number makes the better solubility, while it may cause the increase in burning power, which is disadvantageous to high-speed burning. In contrast, if less carbon number is used, the burning power is relatively low, while it increases the difficulty in preparing dye solution for the worse solubility, and may lower the quality of disc manufacture. So far, there is no appropriate way to solve such problems. However, in the present invention, it has been found that R₅ and R₆ (which are still lower alkyl) with highly polar substituent will maintain a low burning power, and make cyanine dye well soluble in a commonly used polar solvent, such as 2,2,3,3-tetrafluoro-1-propanol (TFP). Such highly polar substituent may be hydroxyl, methylamino, dimethylamino, acetyloxy, propionyloxy, benzoyloxy, carbamoyloxy, dimethylaminoacyloxy, tosyloxy, or tirfluoromethylsulfonyloxy. R₅ and R₆ may be different or identical.

Differently from the azo-metal complex described in lots of reports, the counter ion X⁻ used in the present cyanine dye is an anion selected from Cl⁻, Br⁻, I⁻, ClO₄⁻, BF₄⁻, PF₆⁻, or SbF₆⁻. Y is hydrogen, halogen, methyl, ethyl, phenyl, or alkylamino.

The synthesis pathway to introduce phenyl or phenyl-containing substituent onto R₁ or R₂ (R₃ or R₄) is quite different from conventional synthesis of cyanine. In general, it is impossible to use indole as a starting material. In one embodiment, wherein the one of R₁ and R₂ is benzyl and the other is methyl, synthesis pathway involves the reaction of 2-naphthylhydrazine with benzylacetone under appropriative condition to obtain a benzyl-substituted (on R₁) benzoindole intermediate. Subsequently, methyl may be introduced onto R₂ position of this intermediate by Grignard reaction. After alkylation, the alkyl substituted with hydroxyl or halogen is introduced. A cyanine dye is obtained by performing condensation and bridge formation. The terminal hydroxyl or halogen may further undergo kinds of reaction, such as dehydration and substitution. When R₅ and R₆ are identical, the pathway of dye synthesis may be illustrated by the following equations:

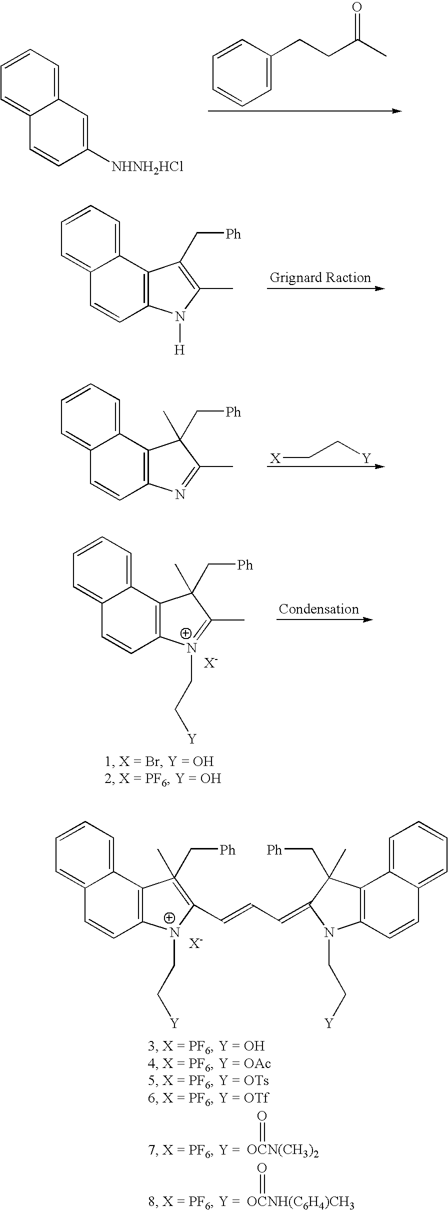

When R₅ and R₆ are different, then condensation and bridge formation need amide compound as the intermediate. The reaction path is described as follow:

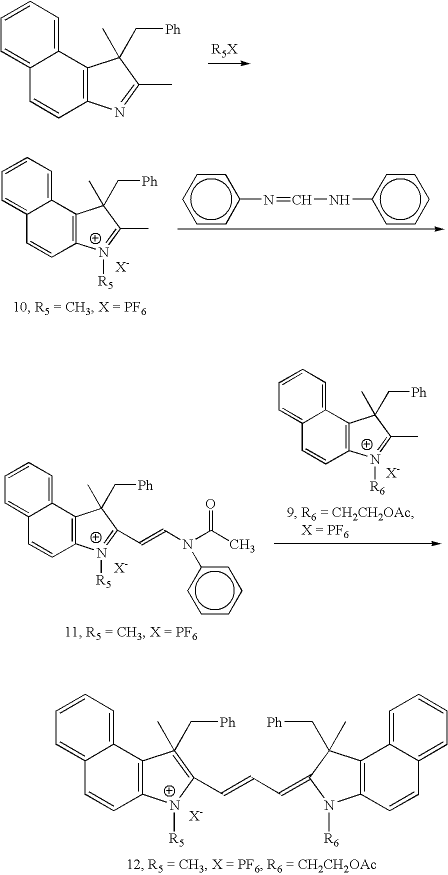

The present invention also provides an optical recording medium, which comprising a transparent substrate, a recording layer, a reflective layer, and a protective layer; wherein the recording layer comprising at least one cyanine dye of the invention as recording material.

Substrate is commonly made of optical transparent resin, which may be selected from polyvinyl chloride resin, epoxy resin, methacrylate resin, polycarbonate resin, and polyolefin resin.

The recording layer may be evenly coated onto the substrate by spin coating method. Briefly, the present cyanine dye is dissolved in a suitable solvent, preferably not beyond 2.5% wt/vol, more preferably at the range of 1.2˜1.8%, with stirring. After filtration, the cyanine dye solution is coated onto the substrate by spin coating method. The thickness of recording layer is between 50 to 300 nm, preferably 80 to 200 nm.

Considering the solvent solubility for certain recording material and the corrosion to the substrate, the suitable solvents for spin coating include halogenated hydrocarbons, such as dichloromethane, chloroform, carbon tetrachloride, trichloroethane, dichloroethane, tetrachloroethane, and dichlorodifluoroethane; alcohols, such as methanol, ethanol, propanol, tetrafluoropropanol, diacetone alcohol, and butanol; and ketones, such as acetone, trifluoroacetone, hexafluoroacetone, and cyclohexanone.

The material of reflective layer is mainly a metal material, such as copper, aluminum, gold or silver, or an alloy material. The reflective layer may be coated on the recording layer by vacuum evaporation or sputtering method. The thickness of reflective layer is generally between 1 to 200 nm.

The protective layer is made of thermosetting resin or UV cross-linked resin, which preferably is transparent. Such resin is coated on the reflective layer by spin coating method to form a protective layer. In general, the thickness of protective layer is between 0.1 to 500 microns, preferably 0.1 to 500 microns.

A double-sided recording medium is obtained by binding two discs prepared as described above with a binder. On the other hand, if the disc prepared as described above is binded to a blank substrate without recording layer, reflective layer, and protective layer coated on, then a single-sided recording medium is obtained.

For the convenience of using, polycarbonate plate is widely used in the manufacture of recording media as substrate, and spin coating method is used for applying recording layer and protective layer.

The present invention will be further defined by reference to the following examples, which are set forth to assist in understanding the invention and should not be construed as specifically limiting the invention. Therefore, any modification or derivative made without departing from the spirit of this invention will be considered to fall within the scope of the invention.

### EXAMPLES

### Example 1

35.0 g of 2-naphthylhydrazine hydrochloride and 29.5 g of sodium acetate were solved in 350.0 ml of hot ethanol. To the solution were added 29.3 g of benzylacetone and 14.0 ml of conc. sulfuric acid, and heated to reflux for 6 hours. After cooling and filtration, 69.6 g of pale pink solid was obtained. The solid was dissolved in 69.6 ml of tetrahydrofuran, and then added to 96.0 ml of methylmagnesium-chloride/tetrahydrofuran solution drop wise. After the reaction of 3 hours, 43.7 g of methane iodide was added, and the mixture was heated for 2 hours. After cooling and filtration, 41.6 g of 1-benzyl-1,2-dimethyl-1H-benzoindole was obtained.

### Example 2

50.1 g of 1-benzyl-1,2-dimethyl-1H-benzoindole and 43.7 g of ethanol bromide in 50.0 ml of acetonitrile were heated to reflux for 8 hours. After cooling, 400.0 ml of ethyl acetate was added. 36.9 g of compound 1 was obtained after filtration. The compound 1 was heated in a mixture of acetonitrile (36.9 mL) and methanol (73.8 mL), and then a mixture of 82.6 g KPF₆/231.3 mL CH₃COCH₃/66.1 mL H O₂ was added, and heated to reflux for 2 hours. After cooling and filtration, 34.3 g of compound 2 was obtained.

### Example 3

54.5 g of compound 2, 114.7 ml of pyridine, and 9.4 g of triethoxymethane were heated for 6 hours. After cooling, 204.3 ml of methanol and 204.3 ml of acetone were added. By filtration, 41.6 g of compound 3 was obtained.

### Example 4

5.0 g of compound 3, 30.0 ml of dichloromethane, and 5.0 ml of pyridine were placed in ice-bath, to which a mixed solution of 10 ml acetyl chloride/0.5 ml dichloromethane was added. After stirring at room temperature for 2 hours, 20.0 ml of methanol was added. By filtration, 4.2 g of compound 4 was obtained.

### Example 5

Repeat the same procedure as described in Example 4, except that acetyl chloride was replaced by 1.4 g of methylsulfonyl chloride. After heating to reflux for 2 hours, 3.9 g of compound 5 was obtained.

### Example 6

Repeat the same procedure as described in Example 4, except that acetyl chloride was replaced by 3.6 g of trifluorosulfonic anhydride, and 5.9 g of compound 6 was obtained.

### Example 7

Repeat the same procedure as described in Example 4, except that acetyl chloride was replaced by 1.4 g of methylcarbamyl chloride. After heating to reflux for 2 hours, 6.1 g of compound 7 was obtained.

### Example 8

5.0 g of compound 3 was dissolved in 10.0 ml of acetone. To the solution 1.0 g of p-tolyl isocyanate was added, and stirred at room temperature for 2 hours. 20.0 ml of methanol and 5.0 ml of water were added, and 3.8 of compound 8 was obtained by filtration.

### Example 9

41.6 g of 1-benzyl-1,2-dimethyl-1H-benzoindole and 43.5 g of methane iodide in 83.2 ml of acetonitrile were heated to reflux for 7 hours. After cooling, 166.4 ml of methanol was added. Subsequently, a mixture of 33.9 g KPF₆/94.8 mL CH₃COCH₃/27.1 mL H O₂ was added, and heated to reflux for 2 hours. After cooling and filtration, 51.1 g of compound 10 was obtained.

### Example 10

8.5 g of compound 10 and 3.9 g of N,N′-diphenyl iminoformamide in 14.9 ml of acetic anhydride were heated to reflux for 7 hours. After cooling, 25.5 ml of ethyl acetate was added. 10.5 g of compound 11 was obtained after filtration.

### Example 11

7.1 g of compound 9, 8.1 g of compound 11, and 3.4 g of sodium acetate in 45.5 ml of ethanol were heated to reflux for 6 hours. After cooling and filtration, 6.9 g of compound 12 was obtained.

### Example 12

A round disc of polycarbonate substrate with thickness of 0.6 mm and diameter of 120 mm, having grooves with depth of about 160 nm, and width (at semi-height) of about 350 nm, and orbital spaces of about 740 nm, was prepared by injection molding.

1.5 g of compound 4 was dissolved in tetrafluoropropanol to form a 1.5% (wt/vol.) recording layer dye solution. The dye solution was passed through 0.2 μm filter, and then coated onto the grooved polycarbonate disc as described above by spin coating method at 400 rpm, the coating speed was gradually increased to 3000 rpm for removing excess dye solution. The formed recording layer was hot air dried at 60° C. for 15 minutes. A silver reflective layer in thickness of 90 nm was sputtered onto the recording layer in a vacuum sputtering system. Subsequently, a UV-hardener (ALCATEL, ATP150 light sensitive resin) was coated onto the silver reflective layer by spin coating method to form a protective layer of about 7 mm. Another polycarbonate substrate of the same size (with thickness of 0.6 mm and diameter of 120 mm) was binded with the disc prepared as described above by UV-hardener and radiation to form a DVDR disc consisting of transparent substrate, recording layer, reflective layer, and protective layer in order.

The blank DVDR disc prepared above (No. 1) was written-in different kinds of information on Pioneer A10 DVD writer at recording speed of 16×, and then tested for 14T signal intensity 114M, reflectivity R14H, etching pit variation Jitter, and parity of inner-code (PI) on an automatic disc-testing system (Pulstec DES-21). The results were listed in Table 1.

### Example 13

Repeat the procedure described in Example 12, except that the compound 4 was replaced by compound 12 to prepare a blank DVDR disc (No. 2). The testing results were listed in Table 1 as follow.

### Example 14

Repeat the procedure described in Example 12, except that the 1.5 g of compound 4 in Example 12 was replaced by 1.0 g of compound 12 and 0.5 g of compound 4 to prepare a blank DVDR disc (No. 3). The testing results were listed in Table 1 as follow.

TABLE 1

| Sample | I14M | R14H (%) | Jitter (%) | PI |
|---|---|---|---|---|
| Disc No. 1 | 77.3 | 49.2 | 7.6 | 218 |
| Disc No. 2 | 75.4 | 53.8 | 7.2 | 105 |
| Disc No. 3 | 76.8 | 52.3 | 7.3 | 121 |

As showed in Table 1, the disc comprising a cyanine dye of the invention with highly polar substituent, such as symmetrical dye (compound 4) used in Disc No. 1, asymmetrical dye (compound 12) used in Disc No. 2, or the combination thereof used in Disc No. 3, could meet the requirement in high-speed (16× or higher) burning, and provide excellent recording quality (such as 114M, R14H, Jitter, and PI).

The above examples are given by way of illustration only, and should not be construed as specifically limiting the scope of present invention. Any variation of the invention described and claimed herein, including the substitution of all equivalents, which would be within the purview of those skilled in the art, is to be considered to fall within the scope of the invention incorporated herein.

1. A cyanine dye of structural formula (I): 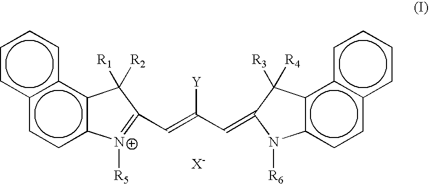
Wherein R₁, R₂, R₃, and R₄ may be different or identical, and each is unsubstituted or substituted C1-C6 alkyl, phenyl, benzyl, or alkylphenyl; R₅, and R₆ is unsubstituted or substituted C1-C4 alkyl, wherein the possible substituent is hydroxyl, alkylamino, alkylacyloxy (—OC(═O)R), alkylaminoacyloxy(—OC(═O)NHR), tosyloxy(—OSO₂C₆H₄CH₃), or tirfluoromethylsulfonyloxy(—OSO₂CF₃); Y is hydrogen, halogen, methyl, ethyl, phenyl, or alkylamino; and counter ion X⁻ is an anion selected from Cl⁻, Br⁻, I⁻, ClO₄⁻, BF₄⁻, PF₆⁻, or SbF₆−.

2. A cyanine dye of claim 1, wherein R₁ and R₃ both are benzyl, and R₂ and R₄ both are CH₃.

3. A cyanine dye of claim 1, wherein R₅ and R₆ both are CH₂CH₂C(═O)CH₃.

4. A use of the cyanine dye described in claim 3, or the mixture thereof, for using as the optical dye in recording layer of optical recording medium.

5. A recording medium comprising a transparent substrate, a recording layer containing organic dye and used for recording data after laser radiation, a reflective layer, and a protective layer, wherein the recording layer comprises a cyanine dye of claim 3, or the mixture thereof, as optical dye.

6. A use of the cyanine dye described in claim 2, or the mixture thereof, for using as the optical dye in recording layer of optical recording medium.

7. A use of the cyanine dye described in claim 1, or the mixture thereof, for using as the optical dye in recording layer of optical recording medium.

8. A recording medium comprising a transparent substrate, a recording layer containing organic dye and used for recording data after laser radiation, a reflective layer, and a protective layer, wherein the recording layer comprises a cyanine dye of claim 2, or the mixture thereof, as optical dye.

9. A recording medium comprising a transparent substrate, a recording layer containing organic dye and used for recording data after laser radiation, a reflective layer, and a protective layer, wherein the recording layer comprises a cyanine dye of claim 1, or the mixture thereof, as optical dye.
